(19) United States
(12) Patent Application Publication
(10) Pub. No.: US 2019/0151271 A1
(43) Pub. Date: May 23, 2019

(54) COMPOSITIONS AND METHODS FOR TREATMENT OF EYE DISEASES

(71) Applicants: The Johns Hopkins University, Baltimore, MD (US); Nacuity Pharmaceuticals, Inc., Fort Worth, TX (US)

(72) Inventors: Peter A. Campochiaro, Baltimore, MD (US); G. Michael Wall, Fort Worth, TX (US)

(21) Appl. No.: 16/192,518

(22) Filed: Nov. 15, 2018

Related U.S. Application Data
(60) Provisional application No. 62/589,397, filed on Nov. 21, 2017.

(51) Int. Cl. A61K 31/198; A61K 9/00; A61P 27/02; A61K 9/107; A61K 47/64

(52) U.S. Cl. CPC A61K 31/198; A61K 9/0048; A61K 47/6415; A61K 9/1075; A61K 9/0097; A61P 27/02

(57) ABSTRACT

The present invention includes compositions and methods for treating an eye disease caused by oxidative damage in a subject in need thereof, the method comprising: identifying or causing the subject to have a compromised blood-retinal barrier; and providing an effective amount of an N-acetylcysteine amide (NACA) sufficient to afford or reach a concentration of NACA in a retina that is greater than the concentration in plasma over the same time period.

### CROSS-REFERENCE TO RELATED APPLICATIONS

This application claims priority to U.S. Provisional Application U.S. Ser. No. 62/589,397, filed Nov. 21, 2017, entitled “Compositions and Methods for Treatment of Eye Diseases” the entire contents of which are incorporated herein by reference.

### STATEMENT OF FEDERALLY FUNDED RESEARCH

None.

### TECHNICAL FIELD OF THE INVENTION

The present invention relates in general to the field of novel methods and compositions for the treatment of eye diseases associated with oxidative damage of the retina.

### BACKGROUND OF THE INVENTION

Without limiting the scope of the invention, its background is described in connection with treating oxidative stress in the eye.

One example of an eye disease is Retinitis Pigmentosa (RP), which is the term used for a genetically heterogeneous group of inherited retinal degenerations. In eye disorders caused by oxidative stress an example of an inciting event is a mutation that leads to the death of rod photoreceptors, initially causing night blindness. Rods are the major consumers of oxygen in the retina and the loss of rods causes an increase in the tissue oxygen level in the outer retina. This activates NADPH oxidase causing accumulation of superoxide radicals in the cytosol and also increases their generation in mitochondria of cones. The excess superoxide radicals overwhelm superoxide dismutase 1 and 2 (SOD1 and SOD2) and cause a chain reaction by which other free radicals are generated including some that are even more damaging than superoxide radicals, such as hydroxyl radicals and peroxynitrite. The free radicals attack proteins, lipids, and DNA causing specific modifications that indicate that oxidative damage has occurred. Oxidative damage to lipids results in lipid hydroperoxides that break down to form 4-hydroxynonenal, malondialdehyde (MDA), and acrolein. The most common modification to proteins from oxidative damage is the formation of carbonyl adducts. Measurements of these markers of oxidative damage, such as MDA or the carbonyl adducts, provide a quantitative assessment of the amount of oxidative damage that has occurred in a tissue. These modifications can impair the function of macromolecules and while there are endogenous repair processes, they are overwhelmed by sever oxidative stress resulting in reduced cellular function and eventually apoptosis. After rods are eliminated from the photoreceptor layer, oxidative stress in the outer retina is severe and leads to gradual cone cell death usually starting in the midperiphery where cone density is low and then spreading peripherally and posteriorly. The posterior spread of cone death results in constriction of the visual field and eventually a central island of vision and its elimination causes blindness.

Currently, there is no approved therapy that stops the evolution of the disease or restores vision. The therapeutic approach is restricted to slowing down the degenerative process by sunlight protection and vitamin A supplementation, treating complications (cataract and macular edema), and helping patients to cope with the social and psychological impact of blindness. Although the Argis II Retinal Prosthesis System was approved by FDA in 2013 as an implanted device to treat adults with several RP, it only produces the sensation of light, thereby helping patients identify the location or movement of objects and people; the device is not disease modifying. Based on studies in animal models described below, NACA is able to treat RP in vivo.

As such, there still exists a need for novel compositions and methods for treatment of eye diseases such as retinitis pigmentosa.

### SUMMARY OF THE INVENTION

In one embodiment, the present invention includes a method for treating an eye disease caused by oxidative damage in an animal or human in need thereof, the method comprising: identifying that the subject has a compromised blood-retinal barrier; and providing an effective amount of an N-acetylcysteine amide (NACA) sufficient to increase the concentration of NACA in a retina that is greater than the concentration in plasma over the same time period. In one aspect, the eye disease is retinitis pigmentosa. In another aspect, the NACA is provided orally, peritoneally, intravenously, dermally, bucally, sublingually, topically, topical ocularly, intraocularly, intravitreally, transmucosally, or by inhalation. In another aspect, the subject is not hypertensive. In another aspect, the blood-retinal barrier is compromised artificially. In another aspect, the blood-retinal barrier is compromised mechanically. In another aspect, the blood-retinal barrier is compromised mechanically with ultrasound, laser, or a penetrator. In another aspect, the blood-retinal barrier is compromised is compromised chemically. In another aspect, the blood-retinal barrier is compromised is compromised chemically with at least one of a microbubble, a toxin, TNF-α, cryotherapy, monomeric C-reactive Protein (mCRP), HIV-1 gp120 glycoprotein. In another aspect, the blood-retinal barrier is compromised with a Toxoplasma gondii toxin. In another aspect, the blood-retinal barrier is reversibly compromised mechanically or chemically. In another aspect, the NACA is administered orally in the form of a tablet, a capsule, a pellet, or a liquid. In another aspect, the NACA is administered topically in the form of a gel, an ointment, a liniment, a lotion, a cream, a pill, a powder, a solution, a suspension, an emulsion, an implant, a sublingual formulation or a suppository. In another aspect, the NACA is administered topically in the form of a solution that is formed by mixing lyophilized NACA with diluent prior to administration. In another aspect, the NACA is administered by an intradermal, intramuscular, intraocular, intravitreal or subcutaneous injection. In another aspect, the dose of NACA is between 1 and 10 mg/day. In another aspect, the dose of NACA is between 10 and 200 mg/day. In another aspect, the 60 and 80 mg/day.

In another embodiment, the present invention includes a method of treating retinitis pigmentosa in a subject comprising: identifying that the subject has a compromised blood-retinal barrier; and providing an effective amount of an N-acetylcysteine amide (NACA) sufficient to increase the concentration of NACA in a retina that is greater than the concentration in plasma over the same time period, wherein penetration of the NACA occurs by passive diffusion and wherein the concentration of NACA is greater than 2.5 μg/gr. In one aspect, the NACA is provided orally, peritoneally, intravenously, dermally, bucally, sublingually, transmucosally, or by inhalation. In another aspect, the subject is not hypertensive. In another aspect, the blood-retinal barrier is compromised artificially. In another aspect, the blood-retinal barrier is compromised mechanically. In another aspect, the blood-retinal barrier is compromised mechanically with ultrasound, laser, or a penetrator. In another aspect, the blood-retinal barrier is compromised is compromised chemically. In another aspect, the blood-retinal barrier is compromised is compromised chemically with at least one of a microbubble, a toxin, TNF-α, cryotherapy, monomeric C-reactive Protein (mCRP), HIV-1 gp120 glycoprotein. In another aspect, the blood-retinal barrier is compromised with a Toxoplasma gondii toxin. In another aspect, the blood-retinal barrier is reversibly compromised mechanically or chemically. In another aspect, the NACA is administered orally in the form of a tablet, a capsule, a pellet, or a liquid. In another aspect, the NACA is administered topically in the form of a gel, an ointment, a liniment, a lotion, a cream, a pill, a powder, a suspension, an emulsion or a suppository. In another aspect, the NACA is administered by an intradermal injection. In another aspect, the dose of NACA is between 10 and 200 mg/day. In another aspect, the dose of NACA is between 60 and 80 mg/day.

In another embodiment, the present invention includes a method for treating an eye disease caused by oxidative damage in a subject in need thereof, the method comprising: reversibly disrupting a blood-retinal barrier; and providing an effective amount of an N-acetylcysteine amide (NACA) sufficient to increase the concentration of NACA in a retina that is greater than the concentration in plasma over the same time period. In one aspect, the blood-retinal barrier is disrupted mechanically. In another aspect, the blood-retinal barrier is disrupted mechanically with ultrasound, laser, or a penetrator. In another aspect, the blood-retinal barrier is disrupted is compromised chemically. In another aspect, the blood-retinal barrier is disrupted is compromised chemically with at least one of a microbubble, a toxin, TNF-α, cryotherapy, monomeric C-reactive Protein (mCRP), HIV-1 gp120 glycoprotein. In another aspect, the blood-retinal barrier is disrupted with a Toxoplasma gondii toxin. In another aspect, the NACA and an agent that reversibly disrupts the blood-retinal barrier are provided concomitantly.

In another embodiment, the present invention includes a composition comprising a an effective amount of an N-acetylcysteine amide (NACA) sufficient to increase the concentration of NACA in a retina that is greater than the concentration in plasma over the same time period and an agent, condition, or effect that disrupts the blood-retinal barrier.

### BRIEF DESCRIPTION OF THE DRAWINGS

For a more complete understanding of the features and advantages of the present invention, reference is now made to the detailed description of the invention along with the accompanying FIG.s and in which:

FIG. 1 is a graph that shows the concentration of NACA in plasma after oral administration in mice.

FIG. 2 is a graph that shows the concentration of NACA in the aqueous humor after oral administration in mice.

FIG. 3 is a graph that shows the concentration of NACA in the vitreous humor after oral administration in mice.

FIG. 4 is a graph that shows the concentration of NACA in the retina after oral administration in mice.

FIG. 5 is a graph that shows a comparison of NACA:NAC Levels in the plasma and retina after oral administration in mice.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

N-Acetylcysteine Amide (NACA). Orally administered N-Acetylcysteine (NAC) has been found to be a particularly effective antioxidant that promotes prolonged cone survival and maintenance of cone function (Schimel et al, 2011 and Dong et al., 2014).

N-Acetylcysteine (NAC) is a well-known thiol-containing antioxidant that has been approved by the FDA as an antidote for acetaminophen intoxication and has been used in the clinic for over 50 years for indications including mucolytic therapy for respiratory conditions with excessive and/or thick mucus production; prevention of radiocontrast-induced nephrotoxicity; treatment of cyclophosphamide-induced hemorrhagic cystitis; and reduction of symptoms of both schizophrenia and bipolar disease (Kelly 1998). NAC's effectiveness has been primarily attributed to its ability to reduce extracellular cystine to cysteine and as a source of sulfhydryl groups (DeVries et al., 1993). However, use of NAC has been limited by several drawbacks, most importantly low membrane penetration and <10% systemic bioavailability for oral administration of oral formulations (Ates et al 2008; Kahns and Bundgaard 1990). Disulfide linkage to proteins and deacetylation of NAC in the intestinal mucosa and lumen are probably the greatest factors in the low oral bioavailability of NAC. Due to its amidated neutral carboxyl group, NACA has increased lipophilicity and, therefore, greater cell permeability and bioavailability than NAC (Atlas et al., 1999).

Treatment of human retinal pigment epithelial cells with NACA protected against oxidative stress-induced cellular injury and death. NACA acts by scavenging existing reactive oxygen species and halting production of reactive oxygen species by reversing lipid peroxidation, and increased the levels of GSH and the phase II detoxification enzyme, glutathione peroxidase. Treatment of mice exposed to phototoxic doses of light with NACA maintained retinal pigment epithelial cell integrity, prevented outer nuclear layer cell death and rescued photoreceptor function. (Schimel et al. 2011).

It has been reported that 7 mg/mL NACA in the drinking water of rd10 mice is more effective than 20 mg/mL NAC in protecting scoptopic and photopic b-waves, and cone density at postnatal day (P)35 and P50. (Dong et al., 2014) NAC is formed rapidly following oral administration of NACA; within 30 minutes following oral administration of 500 mg/kg NACA in rats, the plasma levels of NACA and NAC were 15.07±5.08 and 84.55±6.73 μM. Furthermore, NAC and NACA have been detected in the brain within 30 minutes following oral administration of NACA, suggesting that NACA will cross the blood-retinal barrier. (Tse et al., 2013).

The objective of this study was to determine retinal levels of NACA/NAC after administration of 20 mg/mL NACA in drinking water for 7 days in a mouse model of RP and in wild type mice. This experiment was designed to provide rapid confirmation that NACA and/or NAC penetrate the retina following oral administration and to allow comparison of the tissue levels of NACA and NAC in vitreous and retina to plasma. It also provided a comparison of retinal penetration in mice with RP and wild type mice to determine if the breakdown in the blood-retinal barrier in RP affects the levels of NACA in the retina.

NACA. NACA manufactured by Patheon API Services, Inc. (Florence, S.C.), was used in this study. The certificate of analysis was obtained to confirm the purity of the NACA.

Animals and Treatments. Mice were treated in accordance with the recommendations of the Association for Research in Vision and Ophthalmology. Litters of homozygous rd10/rd10 mice (B6.CXB1-Pde6brd10/J) and wild type C57/BL6 mice (The Jackson Laboratory, Bar Harbor, Me.) were used for these studies.

Starting on post-natal day 28 (P28), animals were given normal drinking water (controls, n=10 for each strain) or water containing 20 mg/ml NACA (n=10 for each strain).

Analysis of NACA and NAC. Upon sacrifice of the mice on P35 (post-natal day 35), retina, aqueous, vitreous and plasma were collected, weighed, and stabilized as quickly as possible and stored at −80° C. prior to shipment to AIT Bioscience, LLC (Indianapolis, Ind.). The qualified bioanalytical method (BAM.0445.01) for the quantitation of total NAC NACA in K₂EDTA was based on derivatization, protein precipitation extraction, and LC-MS/MS instrumental analysis. Any disulfides were reduced to free thiols and subsequently reacted with 2-chloro-1-methylpyridinium iodide (CMPI) to form stable thioethers. The thioether derivatives were detected by LC-MS/MS. Stable label isotope internal standards were used. The method covered a concentration range from 50.0 to 50,000 ng/mL.

NACA Preclinical Study Experiment #1: Evaluation of Retinal Penetration of NACA, as amended July 2017 (Changed Initiation of treatment from P14 to P21 and changed termination point from P21 changed to P35 to match the time points in companion studies.). The research was performed in 3 discrete experiments.

Evaluation of Retinal Penetration of NACA. To determine retinal levels of NACA/NAC after administration of 20 mg/mL NACA in drinking water for 7 days. This experiment was designed to provide rapid confirmation that NACA penetrates the retina following oral administration and to compare levels of NACA and its major metabolite, NAC. in retina, aqueous humor, vitreous humor and plasma. This experiment was also designed to evaluate if the breakdown in the blood-retinal barrier in RP (simulated by the rd10 mice) affects the levels of NACA in the retina by comparing animals with RP to wild type mice.

Rd10 and C57BL/6 wild type mice began treatment at P28 (post-natal day 28), at P35 mice were euthanized and plasma, vitreous and retina samples were sent to AITBioscences (Indianapolis, Ind.) for determination of NACA and NAC levels by a validated method using high-pressure liquid chromatography with tandem mass spectrometric/mass spectrometric detection (LCMS). Experimental groups: (n=10/group). The research was performed in 4 discrete treatment groups:
1. Rd10 allowed to freely drink NACA 20 mg/mL-spiked drinking water with measurements at P35;
2. Rd10 allowed to freely drink untreated water with measurements at P35;
3. C57/BL6 NACA 20 mg/mL in drinking water measurements at P35;
4. C57/BL6 allowed to freely drink untreated water with measurements at P35.

All levels of NACA and NAC were below the limit of quantification of the assay (BLQ) for mice which did not receive NACA in the drinking water. Nearly all measurements of NACA and NAC in aqueous, vitreous and retina were also BLQ and are not discussed.

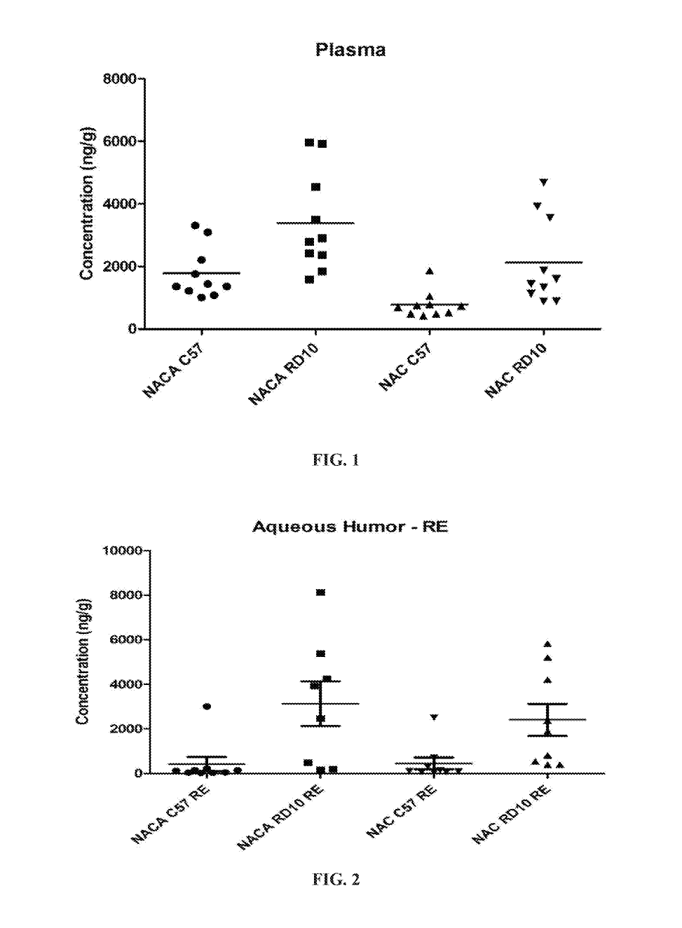

The mean NACA levels in plasma for animals treated with 20 mg/ml in drinking water were greater than the NAC levels in both strains of mice (N=10 for each strain). The mean NACA levels and NAC levels were greater in rd10 mice than in the wild type. (FIG. 1).

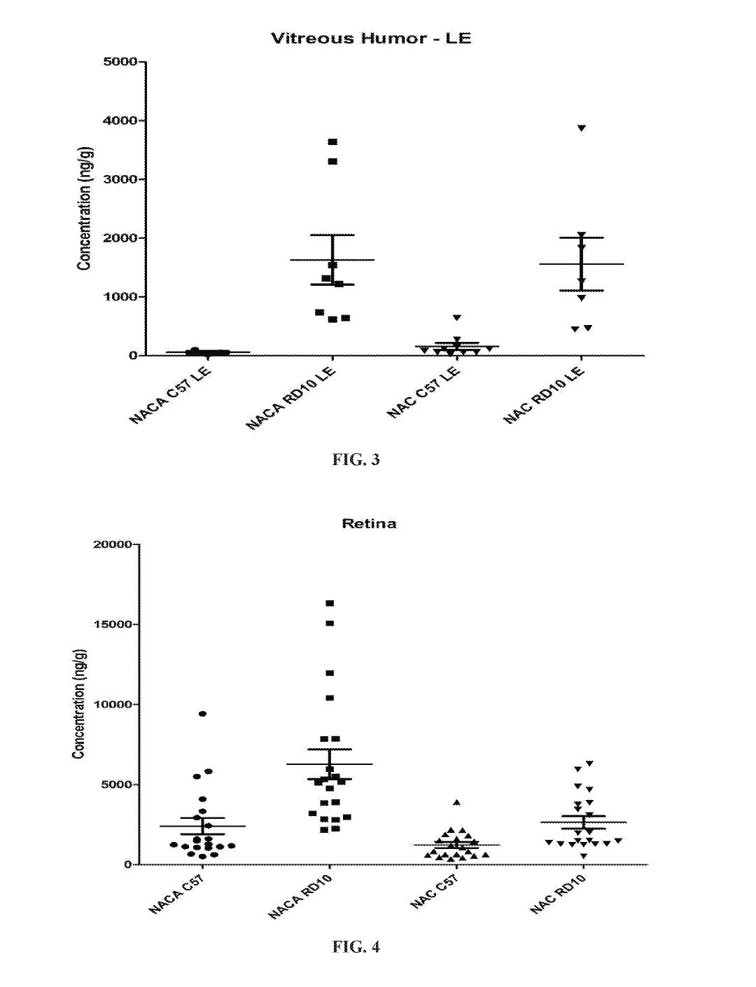

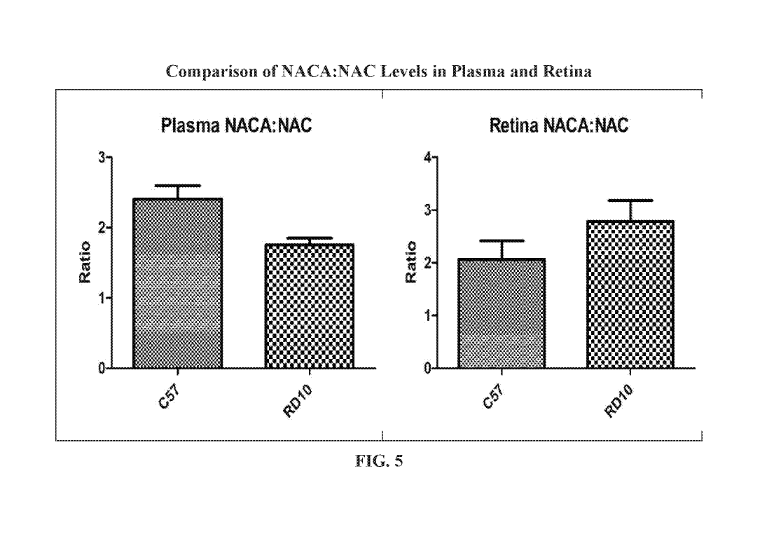

The mean NACA and NAC levels in aqueous humor were lower than in plasma. The mean concentration of NACA in the aqueous humor (N=8 eyes for rd10, N=10 for C57/B16) was also higher in rd10 mice than wild type. (FIG. 2) A similar pattern was observed for the vitreous humor (N=7 or 8 eyes for rd10, N=10 eyes for C57/B16). (FIG. 3) In the retina, NACA and NAC were measurable in all animals treated with NACA (N=20 eyes for each strain). The mean levels of NACA and NAC were higher, in either type of mice, than those observed in plasma (FIG. 4 compared to FIG. 1). The ratio of NACA:NAC in the retina indicates that NACA penetrated retina to a greater extent in rd10 mice than in C57/B16 mice (FIG. 5). That NACA was greater in the retina of rd10 mice proves that the blood retinal barrier of rd10 mice is compromised and greater levels of NACA penetrated to the retina. That retinal levels of NACA were greater in the retina than in plasma suggests that NACA penetrates and is sequestrated in the retina. This is especially surprising given that amidase levels in the retina are greater than plasma (Chastain et al., 2016).

The data from this experiment demonstrate that NACA levels are measurable in the target tissue (especially the retina but also the aqueous humor and vitreous humor) following oral administration of 20 mg/mL in drinking water. It is estimated that mice consume 3-4 mL of water/day, leading to an estimated dose of NACA of 60-80 mg/day. These doses resulted in mean retina levels of approximately 6.5 μg/g in rd10 mice and 2.4 μg/g in wild type mice. It is notable that the ratio of NACA:NAC is greater in the retina of rd10 mice than in C57/B16 mice, suggesting that the disruption of the blood-retinal barrier in RP allows better penetration of NACA.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112 as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Ates B, Abraham L, Ercal N. Antioxidant and free radical scavenging properties of N-acetylcysteine amide (NACA) and comparison with N-acetylcysteine (NAC). Free Radic Res. 2008 April; 42(4):372-7.

Atlas D, Mclamed E, Offen D. Brain targeted low molecular weight hydrophobic antioxidant compounds. U.S. Pat. No. 5,874,468 (1999).

Buss, H., et al., Protein carbonyl measurement by a sensitive ELISA method. Free Radic Biol Med, 1997. 23(3): p. 361-6.

Campochiaro P A, Strauss R W, Lu L, Hafiz G, Wolfson Y, Shah S M, et al. Is There Excess Oxidative Stress and Damage in Eyes of Patients with Retinitis Pigmentosa? Antioxid Redox Signal. 2015 Sep. 1; 23(7):643-8.

Chastain et al. Distribution of topical ocular nepafenac and its active metabolite amfenac to the posterior segment of the eye. Exp Eye Res 2016; 145:58-67

Davies, S. M., et al., Measurements of protein carbonyls, ortho- and meta-tyrosine and oxidative phosphorylation complex activity in mitochondria from young and old rats. Free Radic Biol Med, 2001. 31(2): p. 181-90.

DeVries N, DeFlora S. N-acetyl-l-cysteine. J Cell Biochem Suppl 1993; 17F:270-277.

Dong A, Stevens R, Hackett S, Campochiaro P A. Compared with N-acetylcysteine (NAC), N-Acetylcysteine Amide (NACA) Provides Increased Protection of Cone Function in a Model of Retinitis Pigmentosa. Invest Ophthalmol Vis Sci. 2014 Apr. 30; 55(13):1736-1736.

Kahns A H, Bundgaard H. Prodrugs as drug delivery systems. 107. Synthesis and chemical and enzymatic hydrolysis kinetics of various mono- and diester prodrug of N-acetylcysteine. Int J Pharm 1990; 62:193-205.

Komeima, K., et al., Antioxidants reduce cone cell death in a model of retinitis pigmentosa. Proc Natl Acad Sci USA, 2006. 103(30): p. 11300-5.

Komeima K, Rogers B S, Campochiaro P A. Antioxidants slow photoreceptor cell death in mouse models of retinitis pigmentosa. J Cell Physiol. 2007 December; 213(3):809-15.

Komeima, K., et al., Blockade of neuronal nitric oxide synthase reduces cone cell death in a model of retinitis pigmentosa. Free Radic Biol Med, 2008. 45(6): p. 905-12.

Kelly G S. Clinical applications of N-acetylcysteine. Altern Med Rev J Clin Ther. 1998 April; 3(2):114-27.

Levine, R. L., Carbonyl modified proteins in cellular regulation, aging, and disease. Free Radic Biol Med, 2002. 32(9): p. 790-6.

Schimel A M, Abraham L, Cox D, Sene A, Kraus C, Dace D S, et al. N-acetylcysteine amide (NACA) prevents retinal degeneration by up-regulating reduced glutathione production and reversing lipid peroxidation. Am J Pathol. 2011 May; 178(5):2032-43.

Shen, J., et al., Oxidative damage is a potential cause of cone cell death in retinitis pigmentosa. J Cell Physiol, 2005. 203(3): p. 457-64.

Tse, H. N., et al., High-dose N-acetylcysteinine in stable COPD: the 1-year, double-blind, randomized, placebo-controlled HIACE study. Chest, 2013. 144(1): p. 106-118.

Tuson M, Garanto A, Gonzàlez-Duarte R, Marfany G. Overexpression of CERKL, a gene responsible for retinitis pigmentosa in humans, protects cells from apoptosis induced by oxidative stress. Mol Vis. 2009; 15:168-80.

Usui S, Komeima K, Lee S Y, Jo Y-J, Ueno S, Rogers B S, et al. Increased expression of catalase and superoxide dismutase 2 reduces cone cell death in retinitis pigmentosa. Mol Ther J Am Soc Gene Ther. 2009 May; 17(5):778-86.

Usui S, Oveson B C, Lee S Y, Jo Y-J, Yoshida T, Miki A, et al. NADPH oxidase plays a central role in cone cell death in retinitis pigmentosa. J Neurochem. 2009 August; 110(3):1028-37.

Yu, D. Y., et al., Intraretinal oxygen levels before and after photoreceptor loss in the RCS rat. Invest Ophthalmol Vis Sci, 2000. 41(12): p. 3999-4006.

What is claimed is:

1. A method for treating an eye disease caused by oxidative damage in a subject in need thereof, the method comprising:
identifying that the subject has a compromised blood-retinal barrier; and
providing an effective amount of an N-acetylcysteine amide (NACA) sufficient to increase the concentration of NACA in a retina that is greater than the concentration in plasma over the same time period.

2. The method of claim 1, wherein the eye disease is retinitis pigmentosa.

3. The method of claim 1, wherein the NACA is provided orally, peritoneally, intravenously, dermally, bucally, sublingually, topically, topical ocularly, intraocularly, intravitreally, transmucosally, or by inhalation.

4. The method of claim 1, wherein the subject is not hypertensive.

5. The method of claim 1, wherein the blood-retinal barrier is compromised artificially, chemically, mechanically, ultrasound, laser, or a penetrator.

6. The method of claim 1, wherein the blood-retinal barrier is compromised is compromised chemically with at least one of a microbubble, a toxin, TNF-α, cryotherapy, monomeric C-reactive Protein (mCRP), HIV-1 gp120 glycoprotein, or a Toxoplasma gondii toxin.

7. The method of claim 1, wherein the blood-retinal barrier is reversibly compromised mechanically or chemically.

8. The method of claim 1, wherein the NACA is administered orally in the form of a tablet, a capsule, a pellet, or a liquid; topically in the form of a gel, an ointment, a liniment, a lotion, a cream, a pill, a powder, a solution, a suspension, an emulsion; or as an implant, a sublingual formulation, a suppository; or topically in the form of a solution that is formed by mixing lyophilized NACA with diluent prior to administration.

9. The method of claim 1, wherein the NACA is adapted for administration by an intradermal, intramuscular, intraocular, intravitreal or subcutaneous injection.

10. The method of claim 1, wherein the dose of NACA is between 1 and 10 mg/day, between 10 and 200 mg/day, or 60 and 80 mg/day.

11. A method of treating retinitis pigmentosa in a subject comprising:
identifying that the subject has a compromised blood-retinal barrier; and
providing an effective amount of an N-acetylcysteine amide (NACA) sufficient to increase the concentration of NACA in a retina that is greater than the concentration in plasma over the same time period, wherein penetration of the NACA occurs by passive diffusion and wherein the concentration of NACA is greater than 2.5 μg/gr.

12. The method of claim 11, wherein the NACA is provided orally, peritoneally, intravenously, dermally, bucally, sublingually, transmucosally, or by inhalation.

13. The method of claim 11, wherein the subject is not hypertensive.

14. The method of claim 11, wherein the blood-retinal barrier is compromised artificially, chemically, mechanically, ultrasound, laser, or a penetrator.

15. The method of claim 11, wherein the blood-retinal barrier is compromised is compromised chemically with at least one of a microbubble, a toxin, TNF-α, cryotherapy, monomeric C-reactive Protein (mCRP), HIV-1 gp120 glycoprotein, or a Toxoplasma gondii toxin.

16. The method of claim 11, wherein the blood-retinal barrier is reversibly compromised mechanically or chemically.

17. The method of claim 11, wherein the NACA is administered orally in the form of a tablet, a capsule, a pellet, or a liquid, or topically in the form of a gel, an ointment, a liniment, a lotion, a cream, a pill, a powder, a suspension, an emulsion or a suppository, or by an intradermal injection.

18. The method of claim 11, wherein the dose of NACA is between 10 and 200 mg/day, or between 60 and 80 mg/day.

19. A method for treating an eye disease caused by oxidative damage in a subject in need thereof, the method comprising:
reversibly disrupting a blood-retinal barrier; and
providing an effective amount of an N-acetylcysteine amide (NACA) sufficient to increase the concentration of NACA in a retina that is greater than the concentration in plasma over the same time period.

20. The method of claim 19, wherein the blood-retinal barrier is compromised artificially, chemically, mechanically, ultrasound, laser, or a penetrator.

21. The method of claim 19, wherein the blood-retinal barrier is disrupted is compromised chemically with at least one of a microbubble, a toxin, TNF-α, cryotherapy, monomeric C-reactive Protein (mCRP), HIV-1 gp120 glycoprotein, or a Toxoplasma gondii toxin.

22. The method of claim 19, wherein the NACA and an agent that reversibly disrupts the blood-retinal barrier are provided concomitantly.

23. A composition comprising an effective amount of an N-acetylcysteine amide (NACA) sufficient to increase the concentration of NACA in a retina that is greater than the concentration in plasma over the same time period and an agent, condition, or effect that disrupts the blood-retinal barrier.
